(19) United States
(12) Patent Application Publication
(10) Pub. No.: US 2012/0034165 A1
(43) Pub. Date: Feb. 9, 2012

(54) IMAGING THE CENTRAL NERVOUS SYSTEM WITH PURINERGIC P2X7 RECEPTOR BINDING AGENTS

(76) Inventors: ALexander Jackson, Amersham (GB); Véronique Morisson-Iveson, Amersham (GB)

(21) Appl. No.: 13/263,093

(22) PCT Filed: Apr. 6, 2010
(86) PCT No.: PCT/EP10/54517

§ 371 Date: Oct. 6, 2011

Related U.S. Application Data
(60) Provisional application No. 61/167,893, filed on Apr. 9, 2009.

(30) Foreign Application Priority Data
Apr. 9, 2009 (GB) 0906274.6

(51) Int. Cl. A61K 51/04; B01J 14/00; C07F 7/22; C07D 249/14; C07F 5/02

(52) U.S. Cl. 424/1.89; 548/264.8; 548/110; 548/109; 422/159

(57) ABSTRACT

The present invention provides novel compounds which may be used as in vivo imaging agents. The compounds of the invention are useful in a method to image the expression of P2X7 receptors in a subject, as a means to facilitate the diagnosis of a range of disease states.

### TECHNICAL FIELD OF THE INVENTION

The present invention relates to the field of purinergic P2 receptors. More particularly, the present invention relates to novel purinergic P2X₇ receptor in vivo imaging agents, their production and intermediates thereof. In further detail, the present invention relates to the use of the in vivo imaging agents of the invention in methods to provide information useful in the diagnosis of disease states in which P2X₇ receptor expression is implicated.

### DESCRIPTION OF RELATED ART

The P2X₇ receptor is a cation-selective ion channel directly gated by extracellular ATP (the only known physiological ligand) and a few pharmacological ATP analogues (North 2002 Physiol. Rev. 82.1013-1067). The release of ATP from damaged cells and the subsequent activation of purinergic P2X₇ receptors located on hematopoietic cells (such as microglia, macrophages and lymphocytes) is crucial to the inflammatory cascade (Ferrari D et al 2006 J. Immunol. 176:3877-83). The cation movement associated with the opening of the plasma membrane P2X₇ channel is necessary for the maturation and release of the main pro-inflammatory cytokine, interleukin-1β (IL-1β). While the expression of P2X₇ is low in normal tissue, during inflammation (whether central or peripheral) there is a large increase in P2X₇ reactivity on cells in the surrounding area.

In the central nervous system (CNS), increases in P2X₇ have been characterised following the experimental inducement of stroke (Franke et al 2004 J. Neuropathol. Exp. Neurol. 63:686-99); multiple sclerosis (MS) (Yiangou et al 2006 BMC. Neurol. 6:12); amyotrophic lateral sclerosis (ALS) (Yiangou et al 2006 supra); epilepsy (Rappold et al 2006 Brain Res. 1089:171-8); and, in a transgenic, amyloidic Alzheimer's disease mouse (Parvathenani et al 2003 J. Biol. Chem. 278:13309-17). In the periphery, P2X₇ receptor upregulation has been shown to accompany neuropathic pain (Chessell et al 2005 Pain 114:386-96); polycystic kidney disease (Franco-Martinez et al 2006 Clin. Exp. Immunol. 146:253-61); and, tuberculosis (Hillman et al 2005 Nephron. Exp. Nephrol. 101:e24-30). P2X₇ upregulation has also been shown in a variety of cancers, e.g. cervical, uterine, prostate, breast and skin cancers and leukaemias, both in experimental models and in patients (Feng et al 2006 J. Biol. Chem. 281 17228-37; Greig et al 2003 J. Invest. Deimatol. 121:315-327; Slater et al 2004 Histopathology 44:206-215 Slater et al 2004 Breast Cancer Res. Treat. 83:1-10; Zhang et al 2004 Leuk. Res 28:1313-1322; Li et al 2006 Cancer Epidemiol. Biomarkers Prev. 15:1906-13).

A number of compound classes have been synthesised from different structural backbones to generate therapeutic P2X₇ antagonists. A review of agonists and antagonists acting at the P2X₇ receptor has been published by Baraldi et at (2004 Curr. Topics Med. Chem. 4:1707-17). The compounds disclosed therein are discussed as being potentially useful therapeutic agents. Small molecule P2X₇ binding compounds have also been disclosed in relation to in vivo imaging applications. WO 2007/141267 provides pyrazole derivatives that are P2X₇ antagonists for the treatment of pain, inflammation and neurodegeneration. Isotopically-labelled versions of the compounds are taught to be useful for in vivo imaging by single-photon emission tomography (SPECT) or PET. WO 2007/109154 and WO 2007/109192 disclose bicycloheteroaryl compounds as P2X₇ modulators. Isotopic variants of these comprising ¹¹C, ¹⁸F, ¹⁵O or ¹³N are taught to be useful in PET studies of substrate receptor occupancy. WO 2008/064432 discloses polycyclic compounds for the diagnosis, treatment or monitoring of disorders in which the P2X₇ receptor is implicated. Compounds of WO 2008/064432 that were tested in a P2X₇ receptor functional assay demonstrated that the compounds were antagonists of the P2X₇ receptor. The compounds of WO 2008/064432 may be radiolabelled with an isotope suitable for in vivo imaging, e.g. by SPECT or PET.

There is scope for an alternative in vivo imaging agent suitable for imaging the P2X₇ receptor to facilitate the diagnosis of the broad range of disease states associated with the P2X₇ receptor, in particular those of the central nervous system (CNS).

### SUMMARY OF THE INVENTION

The present invention provides novel compounds which may be used as in vivo imaging agents. The in vivo imaging agents of the invention are particularly useful in a method to image the expression of P2X₇ receptors in the CNS of a subject, as a means to facilitate the diagnosis of a range of disease states.

### DETAILED DESCRIPTION OF THE INVENTION

In one aspect, the present invention provides an in vivo imaging agent suitable for in vivo imaging the central nervous system (CNS) of a subject, wherein said in vivo imaging agent comprises a compound of Formula I, or a salt or solvate thereof, wherein Formula I is defined as follows:

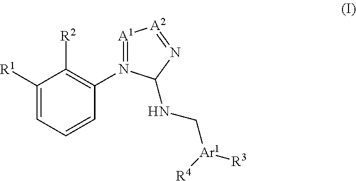

wherein:
R¹ and R² are independently selected from hydrogen, halo, hydroxyl, C₁₋₃ alkyl, C₁₋₃ fluoroalkyl, and C₁₋₃ hydroxyalkyl;
R³ and R⁴ are independently selected from hydrogen, halo, hydroxyl, C₁₋₃ alkyl, C₁₋₃ fluoroalkyl, C₁₋₃ hydroxyalkyl, C₁₋₃ alkyloxy, C₁₋₃ fluoroalkyloxy, C₁₋₃ alkylthio, C₁₋₃ fluoroalkylthio and C₁₋₆ cycloalkyl;
one of A¹ and A² is N and the other is CH;
Ar¹ is a C₅₋₁₂ aryl group optionally comprising 1-3 heteroatoms selected from nitrogen, oxygen and sulfur; and,
wherein any one of R¹, R², R³ and R⁴ as defined comprises an in vivo imaging moiety which is a gamma-emitting radioactive halogen or a positron-emitting radioactive non-metal.

The term “in vivo imaging agent” refers to a compound which can be used to detect a particular physiology or pathophysiology in a living subject by means of its administration to said subject and subsequent detection within said subject, wherein detection is carried out external to said subject.

In order to be “suitable for in vivo imaging of the central nervous system (CNS)” an in vivo imaging agent needs to be able to cross the blood-brain barrier (BBB). The “CNS” is that part of the nervous system of a subject comprising the brain and spinal cord that is covered by the meninges. The generally accepted biophysical/physicochemical models of BBB penetration have as their primary determinants for passive transport: the solute's lipophilicity; hydrogen-bond desolvation potential; pKa/charge; and, molecular size. Typically, a suitable lipophilicity value for a compound to penetrate the BBB would be LogP in the range 1.0-4.5, preferably 2.0-3.5.

The “subject” of the invention is preferably a mammal, most preferably an intact mammalian body in vivo. In an especially preferred embodiment, the subject of the invention is a human.

In the term “salt or solvate thereof”, a suitable salt may be selected from (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycolic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine. A suitable solvate may be selected from those formed with ethanol, water, saline, physiological buffer and glycol.

When a substituent “comprises an in vivo imaging moiety” said substituent either is an in vivo imaging moiety, or said substituent is a chemical group that includes an in vivo imaging moiety, wherein in both cases said in vivo imaging moiety is either a gamma-emitting radioactive halogen or a positron-emitting radioactive non-metal. Such a radioactive isotope is present in the in vivo imaging agent of the invention at a level significantly above the natural abundance level of said radioactive isotope. Such elevated or enriched levels of radioactive isotope are suitably at least 5 times, preferably at least 10 times, most preferably at least 20 times; and ideally either at least 50 times the natural abundance level of the radioactive isotope in question, or present at a level where the level of enrichment of the radioactive isotope in question is 90 to 100%. Examples of chemical groups that comprise an in vivo imaging moiety suitable for the present invention include iodophenyl groups with elevated levels of ¹²³I, CH₃ groups with elevated levels of ¹¹C, and fluoroalkyl groups with elevated levels of ¹⁸F, such that the imaging moiety is the isotopically labelled ¹¹C or ¹⁸F atom within the chemical structure. More detailed discussion of how these and other suitable functional groups are incorporated into the in vivo imaging agents of the invention is given later on in this description.

An “in vivo imaging moiety” allows the compound of the invention to be detected using a suitable imaging modality following its administration to a mammalian body in vivo. Suitable imaging modalities of the present invention include positron-emission tomography (PET) and single-photon emission tomography (SPECT).

When the in vivo imaging moiety is a “gamma-emitting radioactive halogen”, the radiohalogen is suitably chosen from ¹²³I, ¹³¹I or ⁷⁷Br. ¹²⁵I is specifically excluded as it is not suitable for use in in vivo imaging. A preferred gamma-emitting radioactive halogen for in vivo imaging is ¹²³I.

When the imaging moiety is a “positron-emitting radioactive non-metal”, suitable such positron emitters include: ¹¹C, ¹⁷F, ¹⁸F, ⁷⁵Br, ⁷⁶Br or ¹²⁴I. Preferred positron-emitting radioactive non-metals are ¹¹C, ¹⁸F and ¹²⁴I, especially ¹¹C and ¹⁸F, most especially ¹⁸F.

The term “halo” means a substituent selected from fluorine, chlorine, bromine or iodine. “Haloalkyl”, “haloacyl”, “haloalkoxy” and “haloaryl” are alkyl, acyl, alkoxy and aryl groups, respectively, as defined herein, substituted with one or more halo groups. “Fluoroalkyl”, “fluoroalkoxy” and “fluoroalkylthio” are alkyl, alkoxy and alkylthio groups, respectively, as defined herein, substituted with one or more fluoro groups.

Unless otherwise specified, the term “alkyl” alone or in combination, means a straight-chain or branched-chain alkyl radical containing between 1-6 carbon atoms, and preferably between 1 to 3 carbon atoms. Examples of such radicals include, but are not limited to, methyl, ethyl, n-propyl, and isopropyl.

“Hydroxyl” is the group —OH. The term “hydroxyalkyl” represents an alkyl group as defined herein substituted with one or more hydroxyl groups. Preferably a hydroxyalkyl group is of the structure —(CH₂)ₙ—OH wherein n is 1-6.

Unless otherwise specified, the term “alkoxy”, alone or in combination, means an alkyl as defined above which includes an ether radical in the chain (i.e. the group —O—). Examples of suitable alkyl ether radicals include, but are not limited to, methoxy, ethoxy, n-propoxy, isopropoxy, n-butoxy, iso-butoxy, sec-butoxy, tert-butoxy.

The term “thio” means the group —SH. The terms “alkylthio” and “fluoroalkylthio” represent alkyl and fluoroalkyl groups, respectively, as defined herein substituted with one or more thiol groups.

The term “cycloalkyl” refers to an alkyl as defined herein wherein the ends of the chain are joined to faun a cyclic structure.

The term “aryl” refers to aromatic rings or fused aromatic ring systems having 5 to 12 carbon atoms, preferably 5 to 6 carbon atoms, in the ring system, e.g. phenyl or naphthyl. A “heteroatom” is an atom selected from nitrogen, oxygen and sulfur that takes the place of one of the carbon atoms of the aromatic ring. An aryl group comprising one or more heteroatoms is usually termed a “heteroaryl”.

Preferably, R¹ and R² are independently selected from hydrogen, halo, and hydroxyl.

Preferably, R³ and R⁴ are independently selected from hydrogen, hydroxyl, halo, and C₁₋₃ fluoroalkoxy.

Preferably, A¹ is N and A² is CH.

Preferably, Ar¹ is a C₅₋₆ aryl group optionally comprising 1 heteroatom selected from nitrogen, oxygen and sulfur.

In a preferred embodiment, one of R³ and R⁴ comprises the in vivo imaging moiety.

In a most preferred embodiment, the in vivo imaging agent of the invention is a compound of Formula I*:

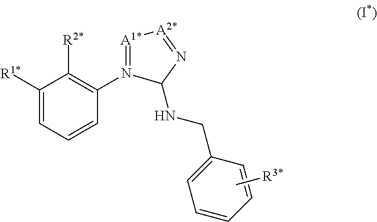

wherein R¹* and R²* are both halo, and R³* is C₁₋₃ alkyl, fluoro, iodo, or C₁₋₃ fluoroalkoxy, and A¹* and A²* are as defined previously for A¹ and A², respectively.

The in vivo imaging agents of the invention are ligands for the P2X₇ receptor, and preferably demonstrate at least 70% inhibition of the function of an agonist to foiin a non-selective pore in HEK.293 cells (see Michel et al, B. J. Pharmacol. 1998; 125: 1194-1201). In terms of binding affinity, a ligand for the P2X₇ receptor has a K_{d} or Kᵢ of between 0.01 and 100 nM, preferably between 0.01 and 10 nM, and most preferably between 0.01 and 1 nM (as measured by: Humphreys et al 1998 Molecular Pharmacology, 54:22-32; Chessell et al 1998 British Journal of Pharmacology, 124: 1314-1320). In conjunction with binding affinity for the P2X₇ receptor, the in vivo imaging agents of the invention preferably have no affinity up to 10 μM for other P2 receptors. The in vivo imaging agent of the invention is preferably an antagonist for the P2X₇ receptor.

The in vivo imaging agent of the invention may be obtained by reaction of a suitable source of the desired in vivo imaging moiety with a non-radioactive precursor compound of Formula Ia:

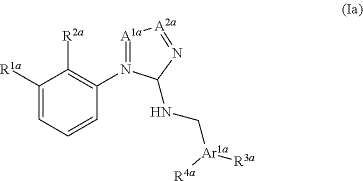

wherein one of R^{1a} to R^{4a} comprises a precursor group and the remainder of R^{1a} to R^{4a} are as defined above for R¹ to R⁴ of Formula I, respectively and optionally comprise a protecting group;
A^{1a} and A^{2a} are as defined above for A¹ and A² of Formula I, respectively;
Ar^{1a} is as defined above for Ar¹ of Formula I.

A “suitable source” of said in vivo imaging moiety means a chemically reactive form of said in vivo imaging moiety. Reaction of the suitable source of said in vivo imaging moiety with the precursor compound preferably leads to the formation of the desired in vivo imaging agent of the invention, without requiring any further steps.

A “precursor compound” comprises an unlabelled, non-radioactive derivative of a compound of Formula I as defined above, i.e. the precursor compound comprises neither a gamma-emitting radioactive halogen nor a positron-emitting radioactive non-metal. The precursor compound is designed so that chemical reaction with a convenient chemical form of the imaging moiety occurs site-specifically; can be conducted in the minimum number of steps (ideally a single step); and without the need for significant purification (ideally no further purification), to give the desired in vivo imaging agent of Formula I as defined herein. Such precursor compounds are synthetic and can conveniently be obtained in good chemical purity. The precursor compound may optionally comprise a protecting group for certain functional groups of the precursor compound.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection, the desired in vivo imaging agent of Formula I as defined herein is obtained. Protecting groups are well known to those skilled in the art and are suitably chosen from, for amine groups: BOC (where BOC is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Fourth Edition, John Wiley & Sons, 2007).

A “precursor group” is a chemical group which reacts with a convenient chemical form of the imaging moiety to incorporate the imaging moiety site-specifically. Suitable such precursor groups are discussed in more detail below. For example, such precursor groups include, but are not limited to, iodo, hydroxyl, nitro, iodonium salt, bromo, mesylate, tosylate, trialkyltin, B(OH)₂, and trialkylammonium salt.

In a preferred embodiment, the precursor compound of Formula Ia is a compound of Formula Ia*:

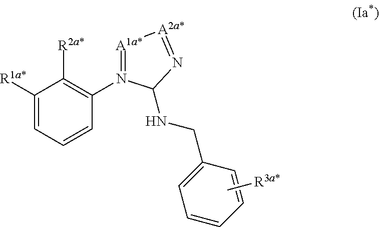

wherein one of R^{1a}* to R^{3a}* comprises a precursor group and wherein the rest of R^{1a}* to R^{1a}* are as defined above for R^{1a} to R^{3a}, respectively, and A^{1a}* and A^{2a}* are as defined above for A^{1a} and A^{2a}, respectively.

Examples of precursor compounds suitable for incorporating representative in vivo imaging moieties of the present invention are now described.

Where the imaging moiety is radioiodine, the in vivo imaging agent as defined herein can be obtained by means of a precursor compound comprising a precursor group which either undergoes electrophilic or nucleophilic iodination or undergoes condensation with a labelled aldehyde or ketone. Examples of the first category are:
(a) organometallic derivatives such as a trialkylstannane (e.g. trimethylstannyl or tributylstannyl), or a trialkylsilane (e.g. trimethylsilyl) or an organoboron compound (e.g. boronate esters or organotrifluoroborates);
(b) a non-radioactive alkyl bromide for halogen exchange or alkyl tosylate, mesylate or triflate for nucleophilic iodination;
(c) aromatic rings activated towards nucleophilic iodination (e.g. aryl iodonium salt aryl diazonium, aryl trialkylammonium salts or nitroaryl derivatives).

Preferred such precursor compounds comprise precursor groups selected from a non-radioactive halogen atom such as an aryl iodide or bromide (to permit radioiodine exchange); an organometallic precursor group (e.g. trialkyltin, trialkylsilyl or organoboron compound); or an organic precursor group such as triazenes, or a precursor group which is a good leaving group for nucleophilic substitution such as an iodonium salt.

Precursor compounds and methods of introducing radioiodine into organic molecules are described by Bolton (J. Lab. Comp. Radiopharm., 2002; 45: 485-528). Suitable boronate ester organoboron compounds and their preparation are described by Kabalka et al (Nucl. Med. Biol., 2002; 29; 841-843, and Nuc. Med Biol. 2003; 30: 369-373). Suitable organotrifluomborates and their preparation are described by Kabalka et al (Nucl. Med. Biol. 2004; 31: 935-938).

Examples of aryl groups to which radioactive iodine can be attached are given below:

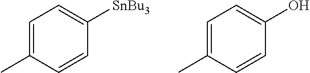

Both contain precursor groups which permit facile radioiodine substitution onto the aromatic ring.

Alternatively, in viva imaging agents containing radioactive iodine can be synthesised by direct iodination via radiohalogen exchange, e.g.

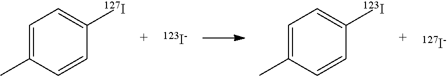

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

Preferably for obtaining in vivo imaging agents of the present invention where the imaging moiety is radioiodine, the precursor compound comprises a precursor group which is an organometallic precursor group, most preferably trialkyltin.

Radiobromination can be achieved by methods similar to those described above for radioiodination. Kabalka and Varma have reviewed various methods for the synthesis of radiohalogenated compounds, including radiobrominated compounds (Tetrahedron 1989; 45(21): 6601-21).

One approach to labelling with ¹¹C is to react a precursor compound which is the desmethylated version of a methylated compound with [¹¹C]methyl iodide. It is also possible to incorporate ¹¹C by reacting a Grignard reagent of the particular hydrocarbon of the desired in vivo imaging agent with [¹¹C]CO₂ to obtain a ¹¹C reagent that reacts with an amine group in the precursor compound to result in the ¹¹C-labelled in vivo imaging agent of interest.

¹¹C could also be introduced as a methyl group on an aromatic ring, in which case the precursor compound would include a precursor group that is a trialkyltin group or a B(OH)₂ group.

As the half-life of ¹¹C is only 20.4 minutes, it is important that the intermediate ¹¹C moieties have high specific activity and, consequently, that they are produced using a reaction process which is as rapid as possible.

A thorough review of such ¹¹C-labelling techniques may be found in Antoni et at “Aspects on the Synthesis of ¹¹C-Labelled Compounds” in Handbook of Radiopharmaceuticals, M. J. Welch and C. S. Redvanly Eds. (2003, John Wiley and Sons).

Preferably for obtaining in vivo imaging agents of the present invention where the imaging moiety is ¹¹C, the precursor compound comprises a precursor group which is trialkyltin group or a B(OH)₂, most preferably trialkyltin.

Radiofluorination may be carried out via direct labelling using the reaction of ¹⁸F-fluoride with a suitable chemical group in a precursor compound having a good leaving group, such as an alkyl bromide, alkyl mesylate or alkyl tosylate. For aryl systems, ¹⁸F-fluoride nucleophilic displacement from an aryl diazonium salt, aryl nitro compound or an aryl quaternary ammonium salt are suitable routes to aryl-¹⁸F derivatives.

Further details of synthetic routes to ¹⁸F-labelled derivatives are described by Bolton (J. Lab. Comp. Radiopharm., 2002; 45: 485-528).

When the in vivo imaging moiety is a radioactive isotope of fluorine the radiofluorine atom may form part of a fluoroalkyl or fluoroalkoxy group, since alkyl fluorides are resistant to in vivo metabolism. Alternatively, the radiofluorine atom may attach via a direct covalent bond to an aromatic ring such as a benzene ring.

¹⁸F can be introduced by O-alkylation of hydroxyl precursor groups with ¹⁸F(CH₂)₃OMs or ¹⁸F(CH₂)₃Br. For aryl systems, ¹⁸F-fluoride nucleophilic displacement from an aryl group of a precursor group which is a diazonium salt, a nitro or a quaternary ammonium salt is a suitable route to obtain an aryl-¹⁸F derivative. Radiofluorination may also be carried out via direct labelling using the reaction of [¹⁸F]-fluoride with a precursor group which is a good leaving group, such as bromide, mesylate, triflate, or tosylate. In this way, the precursor compound may be labeled in one step by reaction with a suitable source of [¹⁸F]-fluoride ion (¹⁸F⁻), which is normally obtained as an aqueous solution from the nuclear reaction ¹⁸O(p,n)¹⁸F and is made reactive by the addition of a cationic counterion and the subsequent removal of water. For this method, the precursor compounds are normally selectively chemically protected so that radiofluorination takes place at a particular site. Suitable protecting groups are those already mentioned previously.

Preferably for obtaining in vivo imaging agents of the present invention where the imaging moiety is ¹⁸F, the precursor compound comprises a precursor group which is a leaving group, most preferably mesylate, triflate, or tosylate.

The preferred and most preferred compounds as defined above in connection with the method of the invention themselves form an additional aspect of the invention.

A particularly preferred in vivo imaging agent of the invention and a precursor compound that was used to obtain it (synthesis described in Example 2) are as follows:

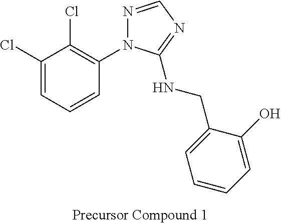

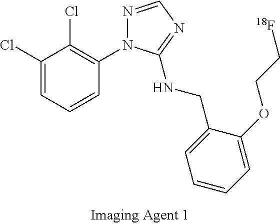

A non-radioactive analogue of the Imaging Agent illustrated in Table I was screened in a P2X₇ receptor functional assay. This assay is described in Example 3 and is based upon the ability of the P2X₇ receptor to form a non-selective pore in P2X₇ transfected HEK.293 cells upon activation with an agonist, thereby allowing dye to permeate the cells. The non selective P2X channel antagonist used as a reference inhibitor for the evaluation of the non-radioactive compound of the invention was pyridoxal-phosphate-6-azophenyl-2′,4′-disulfonate (PPADS), and the results of the assay are provided in Table I above. The non-radioactive analogue of the imaging agent of the invention illustrated in Table I was found to inhibit P2X₇ function at 10 μM and generally at 100 nM concentrations to a similar degree compared to PPADS (the reference compound, which showed 70% inhibition at 10 μM).

The synthetic routes used to obtain Imaging Agent 1 illustrated in Table I, along with its non-radioactive analogue, are provided in Examples 1 and 2. Analogous methods can be used to obtain imaging agents over the whole scope of the claims. Precursors for the synthesis of in vivo imaging agents of the invention may be obtained using methods such as described by Florjancic et al (2008 Bioorg. Med. Chem. Lett., 18: 2089 and references cited therein). Starting compounds and intermediates are either commercially available or described in Florjancic et al (supra) and/or the references cited therein.

To obtain precursor compounds suitable for preparing in vivo imaging agents of the invention where A¹ is N and A² is CH, the following generic reaction scheme may be used:

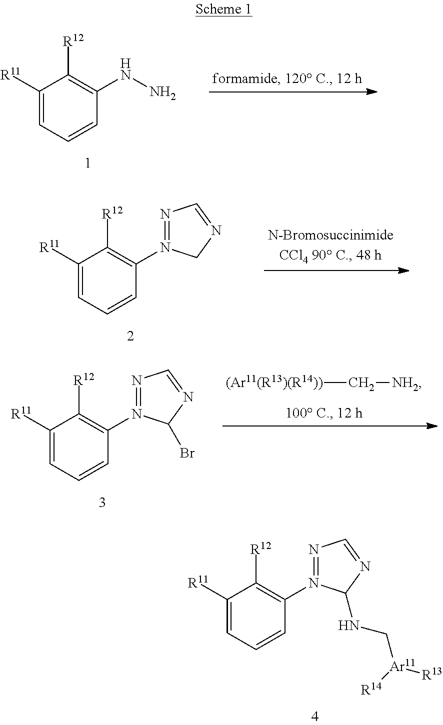

In Scheme 1 above, R¹¹ to R¹⁴ and Ar¹¹ are as defined above for R^{1a} to R^{4a} and Ar^{1a} respectively.

The appropriate phenylhydrazine 1 starting compound is reacted with formamide at elevated temperature to provide the triazole 2, which is in turn brominated to provide intermediate 3. Direct reaction of 3 with an appropriate benzyl amine results in 4.

To obtain precursor compounds suitable for preparing in vivo imaging agents of the invention where A¹ is CH and A² is N, a slightly different generic reaction scheme is used as follows:

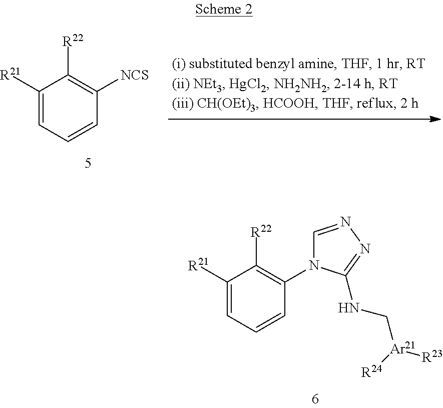

In Scheme 2 above, R²¹ to R²⁴ and Ar²¹ are as defined above for R^{1a} to R^{4a} and Ar^{1a}, respectively, NCS stands for N-Chlorosuccinimide, THF stands for tetrahydrofuran, RT stands for room temperature, and NEt₃ stands for triethylamine.

The starting material for Scheme 2 is the isothiocyanate compound 5. Treatment of 5 with a benzyl amine in THF provides a thiourea intermediate, which, by addition of hydrazine in the presence of base and HgCl₂ gives the corresponding aminoguanidine. This is then heated to reflux in the presence of an orthoformate under acidic conditions to result in the product 6.

The precursor compound for synthesising the imaging agent of the present invention may be conveniently provided as part of a kit, for example for use in a radiopharmacy. Such a kit comprises the precursor compound as defined herein in a sealed container. The sealed container preferably permits maintenance of sterile integrity and/or radioactive safety, plus optionally an inert headspace gas (e.g. nitrogen or argon), whilst permitting addition and withdrawal of solutions by syringe. A preferred sealed container is a septum-sealed vial, wherein the gas-tight closure is crimped on with an overseal (typically of aluminium). Such sealed containers have the additional advantage that the closure can withstand vacuum if desired e.g. to change the headspace gas or degas solutions.

Suitable and preferred embodiments of the precursor compound when employed in the kit of the invention are as already described herein.

The precursor compound for use in the kit may be employed under aseptic manufacture conditions to give the desired sterile, non-pyrogenic material. The precursor compound may alternatively be employed under non-sterile conditions, followed by terminal sterilisation using e.g. gamma-irradiation, autoclaving, dry heat or chemical treatment (e.g. with ethylene oxide). Preferably, the precursor compound is provided in sterile, non-pyrogenic form. Most preferably the sterile, non-pyrogenic precursor compound is provided in the sealed container as described above.

Preferably, all components of the kit are disposable to minimise the possibilities of contamination between runs and to ensure sterility and quality assurance.

In a preferred aspect, the method of synthesis of the present invention is automated. [¹⁸F]-radiotracers in particular are now often conveniently prepared on an automated radiosynthesis apparatus. There are several commercially-available examples of such apparatus, including Tracerlab™ and Fastlab™ (both available from GE Heathcare). The radiochemistry is performed on the automated synthesis apparatus by fitting the cassette to the apparatus. The cassette normally includes fluid pathways, a reaction vessel, and ports for receiving reagent vials as well as any solid-phase extraction cartridges used in post-radiosynthetic clean up steps.

In a yet further aspect, the present invention provides a cassette which can be plugged into a suitably adapted automated synthesiser for the automated synthesis of the in vivo imaging agent of the invention.

The cassette for the automated synthesis of the in vivo imaging agent of the invention comprises.
(i) a vessel containing a precursor compound as defined herein; and
(ii) means for eluting the vessel with a suitable source of an in vivo imaging moiety, said in vivo imaging moiety as defined herein.

The cassette may additionally comprise:
(iii) an ion-exchange cartridge for removal of excess in vivo imaging moiety; and optionally,
(iv) a cartridge for deprotection of the resultant radiolabelled product to form an in vivo imaging agent as defined herein.

The reagents, solvents and other consumables required for the synthesis may also be included together with a data medium, such as a compact disc carrying software, which allows the automated synthesiser to be operated in a way to meet the end user's requirements for concentration, volumes, time of delivery etc.

The in vivo imaging agent of the invention is particularly useful for the assessment by in vivo imaging of the number and/or location of P2X₇ receptors in the CNS of a subject.

In a further aspect therefore, the present invention provides a method of imaging a subject to facilitate the determination of the presence, location and/or amount of P2X₇ receptors in the CNS of a subject, said method comprising the following steps:
(i) providing a subject to whom a detectable quantity of the in vivo imaging agent of the invention has been administered;
(ii) allowing the in vivo imaging agent to bind to P2X₇ receptors in said subject;
(iii) detection of signals emitted by said in vivo imaging agent by an in vivo imaging method; and,
(iv) generation of an image representative of the location and/or amount of said signals.

The method of the invention begins by “providing” a subject to whom a detectable quantity of an in vivo imaging agent of the invention has been administered. Since the ultimate purpose of the method is the provision of a diagnostically-useful image, administration to the subject of the in vivo imaging agent of the invention can be understood to be a preliminary step necessary to facilitate generation of said image.

In an alternative embodiment, step (i) of the method of imaging of the invention can instead be:
(i) administration to said subject of a detectable quantity of the in vivo imaging agent of the invention.

“Administration” of the in vivo imaging agent is preferably carried out parenterally, and most preferably intravenously. The intravenous route represents the most efficient way to deliver the in vivo imaging agent throughout the body of the subject, and therefore across the blood-brain barrier (BBB) and into the central nervous system (CNS) of said subject. Intravenous administration does not represent a substantial physical intervention or a substantial health risk. The in vivo imaging agent of the invention is preferably administered as the pharmaceutical composition of the invention, as defined herein.

A “detectable quantity” of an in vivo imaging agent is an amount that comprises sufficient detectable label to enable signals emitted by the in vivo imaging moiety, following administration of said in vivo imaging agent to said subject, to be detected by the imaging apparatus.

The properties of the in vivo imaging agent of the invention make it suitable for crossing the BBB and binding to P2X₇ receptors within the CNS. Therefore, in the method of the invention the detection and generation steps are carried out on the CNS of said subject, preferably the brain.

The method of the invention may be used to study the location and/or amount of P2X₇ receptor in a healthy subject. However, the method is particularly useful when said subject is known or suspected to have a pathological condition associated with abnormal expression of P2X₇ receptors in the CNS (a “P2X₇ condition”). Such conditions include stroke, multiple sclerosis, amyotrophic lateral sclerosis, epilepsy, and Alzheimer's disease, and the pathophysiology of each comprises neuroinflammation. The term “neuroinflammation” refers to the fundamentally inflammation-like character of microglial and astrocytic responses and actions in the CNS. These responses are central to the pathogenesis and progression of a wide variety of neurological disorders including stroke, epilepsy, Parkinson's disease, multiple sclerosis (MS), amyotrophic lateral sclerosis (ALS), Alzheimer's disease and Huntington's disease. Consequently, the image generated by the method of the invention finds use in providing guidance to a clinician in the diagnosis of such disorders.

In an alternative aspect, the present invention provides a method of diagnosis, comprising steps (i)-(iv) of the in vivo imaging method as defined above, and further comprising the following step:
(ii) evaluating the image generated in step (iv) to diagnose a pathological condition associated with abnormal expression of P2X₇ receptors in the CNS (a “P2X₇ condition”).

The P2X₇ condition of step (v) is any one of those described herein. The evaluating step is carried out by a doctor or a vet, i.e. a person suitably qualified to make a clinical diagnosis. Such a diagnosis represents a deductive medical or veterinary decision, which is made for the purpose of making a decision about whether any treatment is required to restore the subject to health.

In a further alternative embodiment, the method may include the preliminary step of administering the in vivo imaging agent of the invention to the subject. Administration of the in vivo imaging agent of the invention is preferably carried out parenterally, and most preferably intravenously. The intravenous route represents the fastest way of delivering the in vivo imaging agent of the invention across the BBB and into contact with P2X₇ receptors in the CNS. Preferred embodiments of said in vivo imaging agent and subject are as previously defined.

The in vivo imaging agent of the invention is preferably administered as a “radiopharmaceutical composition” which comprises the in vivo imaging agent of Formula I together with a biocompatible carrier, in a form suitable for mammalian administration.

The “biocompatible carrier” is a fluid, especially a liquid, in which the in vivo imaging agent of Formula I is suspended or dissolved, such that the radiopharmaceutical composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such radiopharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose”, and are therefore preferably a disposable or other syringe suitable for clinical use. The pre-filled syringe may optionally be provided with a syringe shield to protect the operator from radioactive dose. Suitable such radiopharmaceutical syringe shields are known in the art and preferably comprise either lead or tungsten.

The radiopharmaceutical composition may be prepared from a kit. Alternatively, they may be prepared under aseptic manufacture conditions to give the desired sterile product. The radiopharmaceutical composition may also be prepared under non-sterile conditions, followed by terminal sterilisation using e.g. gamma-irradiation, autoclaving, dry heat or chemical treatment (e.g. with ethylene oxide).

The method of imaging of the present invention may also be employed as a research tool. For example, for the performance of competition studies which allow the interaction of a drug with P2X₇ receptors to be studied. Such studies include dose-occupancy studies, determination of optimal therapeutic dose, drug candidate selection studies, and determination of P2X₇ receptor distribution in the tissue of interest.

In an alternative embodiment, the method of the invention is effected repeatedly, e.g. before, during and after treatment with a drug to combat a P2X₇ condition. In this way, the effect of said treatment can be monitored over time.

Also provided by the present invention is an in vivo imaging agent of the invention for use in medicine, and in particular for use in a method for the determination of the presence, location and/or amount of inflammation in the CNS of a subject. Suitable and preferred embodiments of said in vivo imaging agent, method and subject are as previously defined.

In a further aspect of the invention, the in vivo imaging agent of the invention may be employed for use in the preparation of a medicament for the determination of the presence, location and/or amount of inflammation in the CNS of a subject. Suitable and preferred embodiments of said in vivo imaging agent and said subject are as previously defined herein.

Detailed methods for the synthesis of particular in vivo imaging agents of the invention are provided in the following non-limiting Examples.

### Brief Description of the Examples

Example 1 describes the synthesis of a non-radioactive analogue of imaging agent 1.

Example 2 describes the synthesis of imaging agent 1.

Example 3 describes the assay used to evaluate binding to the P2X₇ receptor.

### Abbreviations Used in the Examples

AIBN azobisisobutyronitrile
ATP adenosine triphosphate
BOC tert-butoxycarbonyl
Bz-ATP 2′ and 3′-O-(4-benzoylbenzoyl)-ATP
DEAD diethyl azodicarboxylate
DMSO dimethyl sulfoxide
DNA deoxyribonucleic acid
EDCl 1-ethyl-3-(3-dimethylaminopropyl)carbodiimide
HEPES 4-(2-hydroxyethyl)-1-piperazineethanesulfonic acid
HPLC high-performance liquid chromatography
IC50 half maximal inhibitory concentration
LDA lithium diisopropylamide
MeOH methanol
NBS N-bromosuccinimide
PPADs pyrdoxalphosphate-6-azophenyl-2′4′-disulphonic acid
RNA ribonucleic acid
RT room temperature
THF tetrahydrofuran

### EXAMPLES

### Example 1

### Synthesis of a Non-radioactive Analogue of Imaging Agent 1 (1-(2,3-dichlorophenyl)-N-(2-(2-fluoroethoxy)benzyl)-1H-1,2,4-triazol-5-amine)

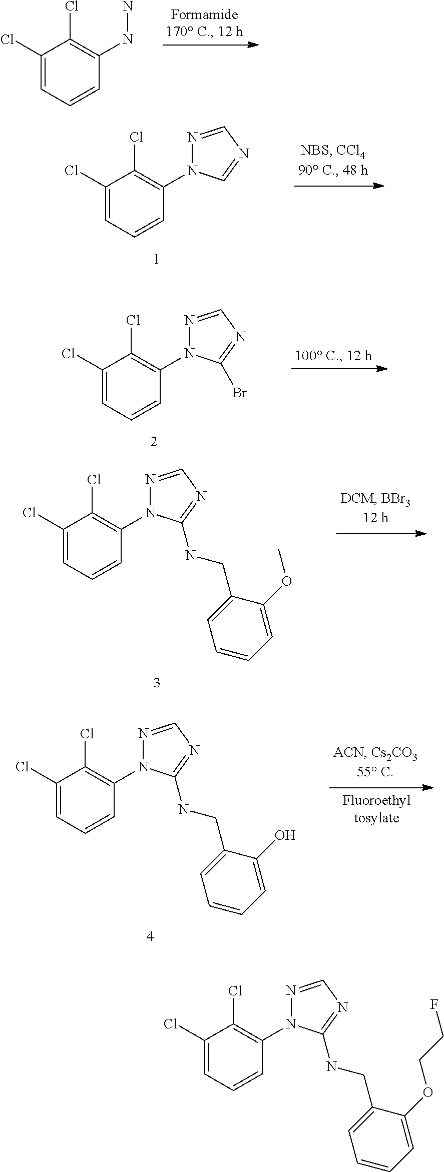

### 1(i) 1-(2,3-Dichlorophenyl)-1H-1,2,4-triazole (1)

Added 2,3-dichlorophenyhydrazine (2.5 g, 14.12 mmol) to an oven dried flask. To this was added formamide (15 mL). The mixture was kept at 170° C. for 12 h. The reaction mass was quenched with water (50 mL) and extracted with ethyl acetate (3×50 mL). The combined organic layers were then washed with water (4×25 mL) and dried over anhydrous sodium sulphate, filtered and evaporated. The crude reaction mass was purified through column chromatography on silica gel using hexane and ethyl acetate as an eluent to give the product (1.8 g, 60% yield) as a white chalky solid.

¹H-NMR: (400 MHz, CDCl₃) δ 8.55 (s, 1H), 8.17 (s, 1H), 7.64 (d, 1H, J=8 Hz), 7.52 (d, 1H, J=8 Hz), 7.40 (t, 1H, J=8 Hz).

### 1(ii) 5-Bromo-1-(2,3-dichlorophenyl)-1H-[1,2,4]-triazole (2)

1-(2,3-Dichlorophenyl)-1H-[1,2,4]-triazole (1), (1.0 g 4.67 mmol) was taken in a round bottom oven dried flask. To this was added anhydrous carbon tetrachloride (15 mL) followed by freshly crystallized NBS (1.1 g, 6.18 mmol) and a catalytic amount of AIBN. The reaction was then heated to reflux at 90° C. for about 48 h. The carbon tetrachloride was removed under reduced pressure and the reaction mass was purified by column chromatography on silica gel using hexane and ethyl acetate as the solvent to give the product 2 (900 mg, 65% yield).

¹H-NMR: (400 MHz, CDCl₃) δ 8.10 (s, 1H), 7.72 (d, 1H, J=8 Hz), 7.41 (q, 2H, J=8 Hz).

### 1(iii) 1-(2,3-Dichlorophenyl)-N-(2-methoxybenzyl)-1H-[1,2,4]-triazol-5-amine (3)

Both the starting material 2 (500 mg, 1.7 mmol) and 2-methoxybenzylamine (0.5 mL) were taken in a round bottom flask. and heated to reflux at 100° C. for 12 h. The reaction was then quenched with water (20 mL) and extracted with ethyl acetate (3×20 mL). The combined extracts were washed with brine (15 mL), dried over anhydrous sodium sulphate, filtered and evaporated. The product was isolated by column chromatography on silica gel using hexane and ethyl acetate as eluent to give 3 (500 mg, 84% yield).

¹H-NMR. (400 MHz, CDCl₃) δ 7.71 (s, 1H), 7.62 (t, 1H, J=4 Hz), 7.25-7.41 (m, 4H), 6.94 (t, 1H, J=8 Hz), 6.88 (d, 1H, J=8 Hz), 4.68 (s, 1H), 4.60 (s, 2H), 3.8 (s, 3H).

### 1(iv) 2-((1-(2,3-Dichlorophenyl)-1H-[1,2,4]-triazol-5-yl-amino)methyl)phenol (4)

1-(2,3-Dichlorophenyl)-N-(2-methoxybenzyl)-1H-[1,2,4]-triazol-5-amine (3) (500 mg, 1.4 mmol) was dissolved in anhydrous dichloromethane (5 mL) in a dried flask. The reaction mass was then cooled to −78° C. and stirred for 15 minutes. Boron tribromide (0.4 ml, 1.6 mmol) was then added while maintaining the same temperature. The reaction was allowed to come to room temperature and stirred for a total of about 12 h before being quenched with water (20 mL slow addition) and extracted with dichloromethane (3×20 mL) and purified by column chromatography on silica gel using hexane-ethyl acetate eluent to give the product (160 mg, 33% yield).

¹H-NMR: (400 MHz, CDCl₃) δ 7.72 (s, 1H), 7.65 (m, 1H), 7.38 (d, 2H, J=4 Hz), 7.25 (t, 1H, J=8 Hz), 7.14 (d, 1H, J=8 Hz), 7.01 (d, 1H, J=8 Hz), 6.88 (t, 1H, J=8 Hz), 4.76 (s, 1H), 4.50 (s, 2H). 1(v) 1-(2,3-dichlorophenyl)-N-(2-(2-fluoroethoxy)benzyl)-1H-[1,2,4]-triazol-5-amine) (Non-radioactive Analogue of Imaging Agent 1)

4 (160 mg, 0.48 mmol) was dissolved in acetonitrile (2 mL) and cesium carbonate (1.2 eqv) was added and mixture then stirred for 15 minutes at room temperature. Fluoroethyl tosylate (1.1 eqv) was added to this mixture and the reaction heated at 55° C. for 12 h. Acetonitrile was then removed under reduced pressure and the residue partitioned between ethyl acetate and water. The organic layer was then concentrated and purified by column chromatography on silica gel using hexane and ethyl acetate as eluent to give the desired product (96 mg, 53% yield).

¹H-NMR. (400 MHz, CDCl₃) δ 7.50-7.82 (m, 2H), 7.11-7.49 (m, 5H), 6.98 (t, 1H, J=8 Hz), 6.84 (d, 1H, J=8 Hz), 4.62 (m, 5H), 4.17 (m, 2H).

### Example 2

### Synthesis of Imaging Agent 1

Imaging Agent 1 is obtained using the method as described in Example 1 except that 4 is reacted with [¹⁸F]-Fluoroethyl tosylate (synthesised e.g. as described by Bauman et al Tetrahedron Letts. 2003; 44: 9165-7) in acetonitrile in the presence of potassium carbonate and Kryptofix.

### Example 3

### Pore-Forming Assay to Determine P2X₇ Binding

The assay method used was based on the ability of the DNA binding dye, Yo Pro-1 (quinolinium, 4[3-methyl-2(3H)-benzoxazolylidene)methyl]-1-[3-(trimethyl-ammonio)propyl]-dioxide) to enter through the dilated or “large pore form” of the P2X₇ receptor and to bind to intracellular DNA/RNA whereupon it increases fluorescence intensity. Yo Pro-1 was therefore used to quantify inhibition of P2X₇ function. This assay was based on the methods published by Michel et al., (B.J. Pharmacol 1998; 125: 1194-1201).

Initially, HEK.293 cells were transiently transfected using LipofectamineTMLTX (Invitrogen) for 72 hrs with P2X₇ cDNA. 48 hours prior to use the cells were seeded into poly-D-lysine coated 96-well black-walled, clear bottomed plates, at a density of 30,000 cells/well. Stock solutions of test compound were prepared at a concentration of 40 mM in 100% DMSO.

Following the 48 hour incubation the culture medium was removed from the transfected cells, the cells were washed once and placed in pre-warmed sucrose assay buffer (Sucrose: 280 mM, KCL 5 mM, CaCl₂: 0.5 mM, glucose: 10 mM, HEPES: 10 mM, N-methyl-D-glucamine: 10 mM; pH7.4). The test compounds were added to the plate at a concentration of 10 μM and 100 nM in triplicate and incubated at 37° C. for 30 minutes. The final DMSO concentration in the assay was 1%. After this time Yo Pro-1 dye and Bz-ATP solution was added at concentrations of 1 μM and 30 μM respectively for 60 minutes at 37° C. The fluorescence was then read at 485 nM excitation and 530 nM emission.

The non-selective P2X channel antagonist pyrdoxalphosphate-6-azophenyl-2′4′-disulphonic acid (PPADS) was used as a reference inhibitor in the assay. A dose-response to PPADS was performed on the assay plate using a starting concentration of 200 μM followed by a 1 in 6 serial dilution covering the concentration range 200 μM to 0.4 nM. For each compound data set, a percentage inhibition value was calculated based on the three assay points generated. For imaging agent 1% inhibition was found to be 77.0 at 10 μM and 68.0 at 100 μM

What is claimed is:

1. An in vivo imaging agent comprising a compound of Formula I: 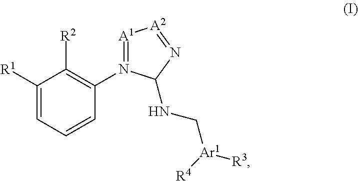
or a salt or solvate thereof, wherein:
R¹ and R² are independently selected from hydrogen, halo, hydroxyl, C₁₋₃ alkyl, C₁₋₃ fluoroalkyl, and C₁₋₃ hydroxyalkyl;
R³ and R⁴ are independently selected from hydrogen, halo, hydroxyl, C₁₋₃ alkyl, C₁₋₃ fluoroalkyl, C₁₋₃ hydroxyalkyl, C₁₋₃ alkyloxy, C₁₋₃ fluoroalkoxy, C₁₋₃ alkylthio, C₁₋₃ fluoroalkylthio and C₁₋₆ cycloalkyl;
one of A¹ and A² is N and the other is CH;
Ar¹ is a C₅₋₁₂ aryl group optionally comprising 1-3 heteroatoms selected from nitrogen, oxygen and sulfur; and,
wherein any one of R¹, R², R³ and R⁴ as defined comprises an in vivo imaging moiety which is a gamma-emitting radioactive halogen or a positron-emitting radioactive non-metal.

2. The in vivo imaging agent as defined in claim 1 wherein R¹ and R² are independently selected from hydrogen, halo, and hydroxyl.

3. The in vivo imaging agent as defined in claim 1 wherein R³ and R⁴ are independently selected from hydrogen, hydroxyl, halo, and C₁₋₃ fluoroalkoxy.

4. The in vivo imaging agent as defined in claim 1 wherein A¹ is N and A² is CH.

5. The in vivo imaging agent as defined in claim 1 wherein Ar¹ is a C₅₋₆ aryl group optionally comprising 1 heteroatom selected from nitrogen, oxygen and sulfur.

6. The in vivo imaging agent as defined in claim 1 wherein one of R³ and R⁴ comprises said in vivo imaging moiety.

7. The in vivo imaging agent as defined in claim 1, wherein said compound of Formula I is a compound of Formula I*: 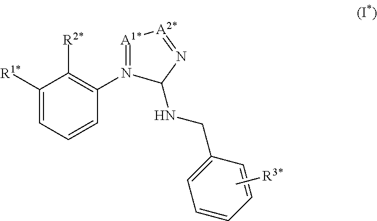
wherein R¹* and R²* are both halo, and R³* is C₁₋₃ alkyl, fluoro, iodo, or C₁₋₃ fluoroalkoxy, and one of A¹* and A²* is N and the other is CH.

8. The in vivo imaging agent as defined in claim 1 wherein said in vivo imaging moiety is selected from ¹²³I, ¹¹C and ¹⁸F.

9. The in vivo imaging agent as defined in claim 8 wherein said in vivo imaging moiety is ¹⁸F.

10. A method for the synthesis of an in vivo imaging agent as defined in claim 1, wherein said method comprises reaction of a suitable source of said in vivo imaging moiety with a non-radioactive precursor compound of Formula Ia: 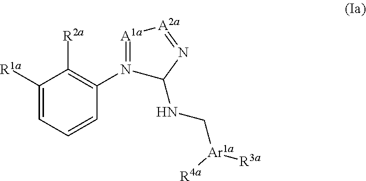
wherein one of R^{1a} to R^{4a} comprises a precursor group and the remainder of R^{1a} to R^{4a} are independently selected from hydrogen, halo, hydroxyl, C₁₋₃ alkyl, C₁₋₃ fluoroalkyl, C₁₋₃ hydroxyalkyl, C₁₋₃ alkyloxy, C₁₋₃ fluoroalkoxy, C₁₋₃ alkylthio, C₁₋₃ fluoroalkylthio and C₁₋₆ cycloalkyl, and optionally comprise a protecting group;
one of A^{1a} and A^{2a} is N and the other is CH; and, Ar^{1a} is a C₅₋₁₂ aryl group optionally comprising 1-3 heteroatoms selected from nitrogen, oxygen and sulphur.

11. The method as defined in claim 10 wherein said precursor compound of Formula Ia is a compound of Formula Ia*: 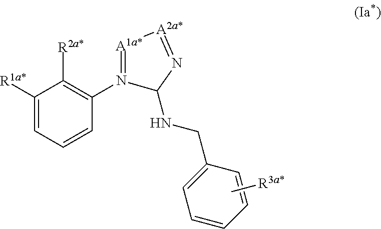
wherein one of R^{1a}* to R^{3a}* comprises a precursor group and wherein the rest of R^{1a}* to R^{3a}* are independently selected from hydrogen, halo, hydroxyl, C₁₋₃ alkyl, C₁₋₃ fluoroalkyl, C₁₋₃ hydroxyalkyl, C₁₋₃ alkyloxy, C₁₋₃ fluoroalkoxy, C₁₋₃ alkylthio, C₁₋₃ fluoroalkylthio and C₁₋₆ cycloalkyl,
and one of A^{1a}* and A^{2a}* is N and the other is CH.

12. The method as defined in claim 10 wherein said method is automated.

13. The method as defined in claim 10 wherein said precursor group is selected from a trialkyltin group, B(OH)₂, mesylate, triflate, or tosylate.

14. A precursor compound selected from a trialkyltin group, B(OH)₂, mesylate, triflate, and tosylate.

15. A cassette comprising:
(i) a vessel containing a precursor compound of Formula Ia: 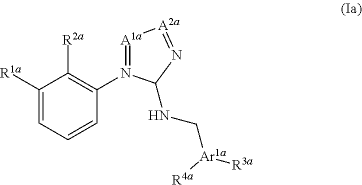
wherein one of R^{1a} to R^{4a} comprises a precursor group and the remainder of R^{1a} to R^{4a} are independently selected from hydrogen, halo, hydroxyl, C₁₋₃ alkyl, C₁₋₃ fluoroalkyl, C₁₋₃ hydroxyalkyl, C₁₋₃ alkyloxy, C₁₋₃ fluoroalkoxy, C₁₋₃ alkylthio, C₁₋₃ fluoroalkylthio and C₁₋₆ cycloalkyl, and optionally comprise a protecting group; one of A^{1a} and A^{2a} is N and the other is CH; and, Ar^{1a} is a C₅₋₁₂ aryl group optionally comprising 1-3 heteroatoms selected from nitrogen, oxygen and sulphur; and
(ii) means for eluting the vessel with a suitable source of an in vivo imaging moiety, which is a gamma-emitting radioactive halogen or a positron-emitting radioactive non-metal.

16. The cassette as defined in claim 15 which additionally comprises:
(iii) an ion-exchange cartridge for removal of excess in vivo imaging moiety; and optionally,
(iv) a cartridge for deprotection of the resultant radiolabelled product to form an in vivo imaging agent comprising a compound of Formula I: 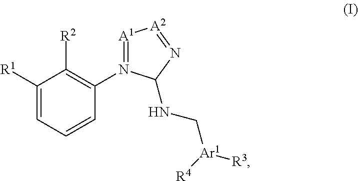
or a salt or solvate thereof, wherein:
R¹ and R² are independently selected from hydrogen, halo, hydroxyl, C₁₋₃ alkyl, C₁₋₃ fluoroalkyl, and C₁₋₃ hydroxyalkyl;
R³ and R⁴ are independently selected from hydrogen, halo, hydroxyl, C₁₋₃ alkyl, C₁₋₃ fluoroalkyl, C₁₋₃ hydroxyalkyl, C₁₋₃ alkyloxy, C₁₋₃ fluoroalkoxy, C₁₋₃ alkylthio, C₁₋₃ fluoroalkylthio and C₁₋₆ cycloalkyl;
one of A¹ and A² is N and the other is CH;
Ar¹ is a C₅₋₁₂ aryl group optionally comprising 1-3 heteroatoms selected from nitrogen, oxygen and sulfur; and,
wherein any one of R¹, R², R³ and R⁴ as defined comprises an in vivo imaging moiety which is a gamma-emitting radioactive halogen or a positron-emitting radioactive non-metal.

17. A radiopharmaceutical composition which comprises the in vivo imaging agent as defined in claim 1, together with a biocompatible carrier, in a form suitable for mammalian administration.

18. A method of in vivo imaging a subject to facilitate the determination of the presence, location and/or amount of P2X₇ receptors in the CNS of a subject, said method comprising the following steps:
(i) providing a subject to whom a detectable quantity of an in vivo imaging agent as defined in claim 1 has been administered;
(ii) allowing the administered in vivo imaging agent to bind to P2X₇ receptors in said subject;
(iii) detection of signals emitted by said in vivo imaging agent by an in vivo imaging method; and,
(iv) generation of an image representative of the location and/or amount of said signals.

19. The method as defined in claim 18 wherein said subject is a mammalian body.

20. The method as defined in claim 18 wherein said subject is known or suspected to have a pathological condition associated with abnormal expression of P2X₇ receptors in the CNS.

21. A method of diagnosis comprising the method as defined in claim 18, and further comprising the following step:
(v) evaluating the image generated in step (iv) to diagnose a pathological condition associated with abnormal expression of P2X₇ receptors in the CNS.

22. (canceled)

23. (canceled)
